(19) United States
(12) Patent Application Publication
(10) Pub. No.: US 2017/0232235 A1
(43) Pub. Date: Aug. 17, 2017

(54) PROTECTIVE BARRIER FOR FLUID CONDUCTING LINES

(71) Applicant: Adam M. Au, Miami Beach, FL (US)

(72) Inventor: Adam M. Au, Miami Beach, FL (US)

(21) Appl. No.: 15/219,971

(22) Filed: Jul. 26, 2016

Related U.S. Application Data
(63) Continuation-in-part of application No. 15/045,076, filed on Feb. 16, 2016, now abandoned.

(51) Int. Cl. A61M 25/02; A61M 25/00

(52) U.S. Cl. CPC A61M 25/02; A61M 25/0023; A61M 2025/0025; A61M 2025/0246; A61M 2025/0266

(57) ABSTRACT

A protective barrier is provided. The protective barrier includes an elongated tube, which may include a protective gasket shape or boxed shaped apparatus. The elongated tube includes an inner surface, an outer surface, a first rim forming an entrance and a second rim forming an exit. A length of the elongated tube is from the first rim to the second rim. Tape attaches the elongated tube to the user's skin at an entry point of the fluid conducting lines. A first side edge and a second side edge are formed along the length of the elongated tube, forming a slit. The first side edge and the second side edge are biased to overlap one another. A disinfectant layer such as an anti-bacterial, anti-viral, and/or anti-septic, is disposed on the inner surface of the elongated tube.

### CROSS-REFERENCE TO RELATED APPLICATION

This application is a continuation-in-part of U.S. non-provisional application Ser. No. 15/045,076, filed Feb. 16, 2016, the contents of which are herein incorporated by reference.

### BACKGROUND OF THE INVENTION

The present invention relates to fluid conducting lines for patients and, more particularly, to a protective barrier for fluid conducting lines.

In daily patient care and in the field of medicine and biotechnology, several fluid conducting lines are used for vascular access, drainage ducts of body organ systems, or as extensions of dysfunctional biological fluid conducting systems, that become modes of infectious transmission into the blood stream and organs. These lines are, but not limited to Intracranial Monitoring Lines, Internal Jugular Central Lines, Subclavian Central Lines, Tracheostomy Valves, Port-A-Catheters, Dialysis and tunneled Access Lines, Peripherally Inserted Central Catheters or Lines, Mid-Lines, Chest Tubes, PleurX Catheters, Biliary Drainage Lines, JP drains, Epidural Access Catheters, Nephrostomy tubes, Percutaneous Gastrojejunostomy Tubes, Suprapubic Catheters, Intravenous Access Lines, Radial Blood Pressure Monitoring Lines, Foley Catheters, Indwelling Paracentesis/Peritoneal Fluid Drainage Lines, Femoral Central Lines, Wound Vacuums, Perianal or body wall Abscess fistula drainage lines, Pulmonary Capillary wedge lines, Dobhoff enteral tubes, and cardiac pump/device exiting lines.

The immediate contacting surface of these accesses to the cutaneous surface creates an infective biological milieu for entry into body organs and the seeding of blood stream with bacteria, fungi and viruses. This infectious biofilm mechanism of entry is not at the lumen, but located on the external cylindrical periphery, and via chemotaxis is able to enter the blood stream. This mechanism is worsened by prolong indwelling of the access line and also by routine contact with sheets, bed rails and the like.

As can be seen, there is a need for an apparatus that reduces the incidence of microburden and biofilm surface contamination of the devices mentioned above in adult and pediatric intensive care units, emergency service department, inpatient hospital environments, long-term acute care units, and home health services.

### SUMMARY OF THE INVENTION

In one aspect of the present invention, a protective barrier comprises: an elongated tube comprising an inner surface, an outer surface, a first rim forming an entrance and a second rim forming an exit, wherein a length of the elongated tube is from the first rim to the second rim; a tape operable to secure the elongated tube to a skin of a user; a first side edge and a second side edge formed along the length forming a slit, wherein the first side edge and the second side edge are biased to overlap; and a disinfectant layer disposed on the inner surface of the elongated tube.

These and other features, aspects and advantages of the present invention will become better understood with reference to the following drawings, description and claims.

### BRIEF DESCRIPTION OF THE DRAWINGS

FIG. 1A is a perspective partial cut away view of an embodiment of the present invention in use;

FIG. 1B is a front partial cut away view of an embodiment of the present invention in use;

FIG. 2A is a perspective partial cut away view of an embodiment of the present invention in use;

FIG. 2B is a front partial cut away view of an embodiment of the present invention in use;

FIG. 3 is a cut away view taken along line 3-3 of FIG. 2B;

FIG. 4 is a top view of an adapter head of an embodiment of the present invention;

FIG. 5 is a cut away view taken along line 5-5 of FIG. 4;

FIG. 6 is a cross sectional view of an embodiment of the present invention in use;

FIG. 7A-7H are cross sectional views of alternate shapes of the present invention;

FIG. 8A-8D are cross sectional views of alternate shapes of the present invention;

FIG. 9 is a front partial cutaway view of an embodiment of the present invention; and

FIG. 10 is a front view of an embodiment of the present invention in use;

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a structural disinfecting barrier installed at the periphery of the immediate contact areas of the arterial, venous, and fluid conducting lines. In addition to forming a physical barrier, the present invention further includes a film including disinfectants disposed at the inner surface of the lumen where the actual lines transverse. The protective barrier prevents the entry point from becoming infected.

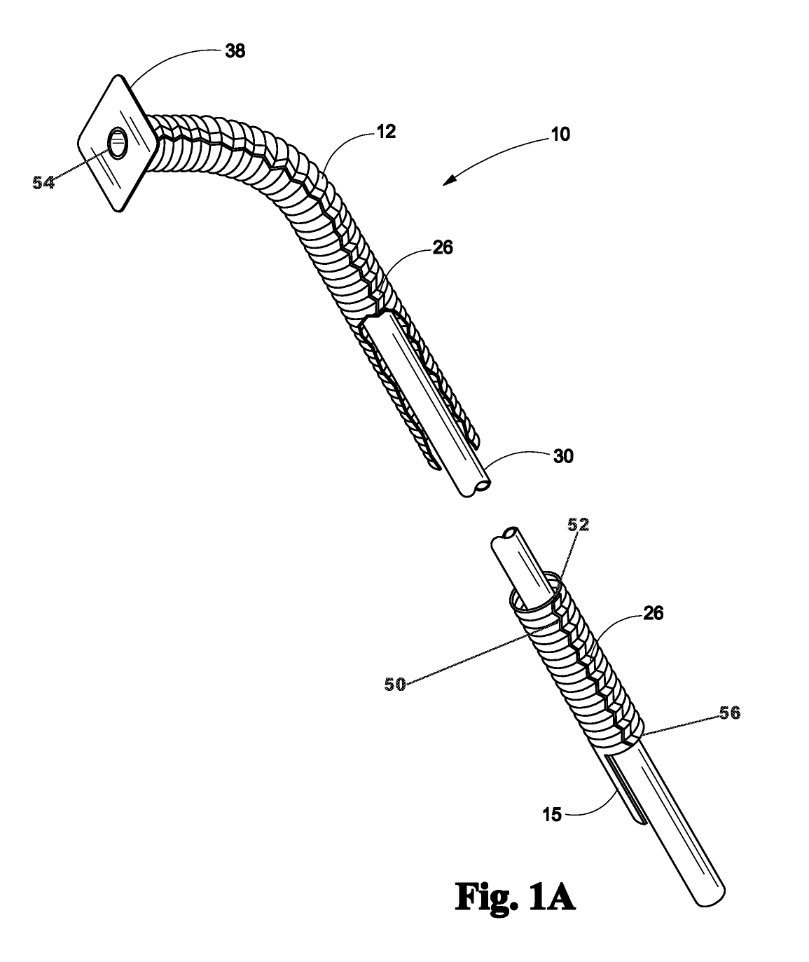

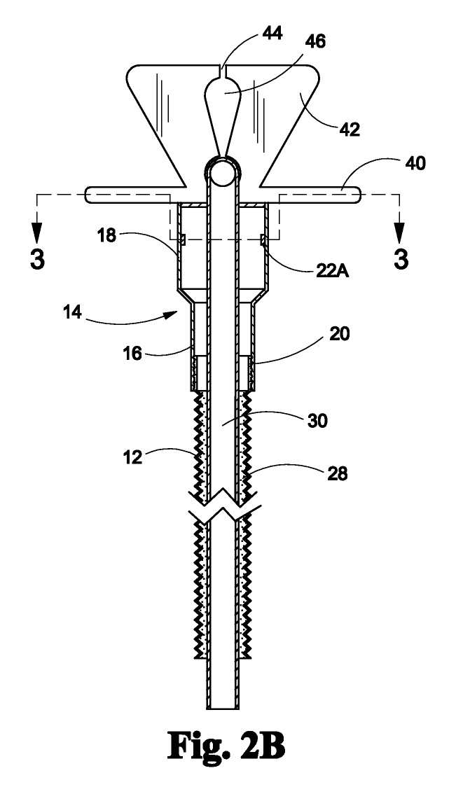

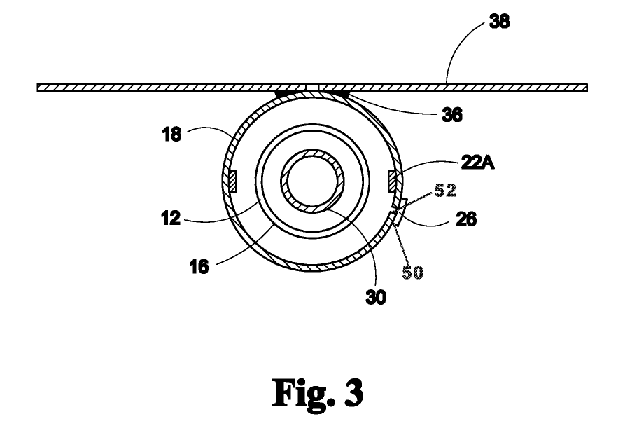

Referring to FIGS. 1 through 8, the present invention includes a protective barrier. The protective barrier includes an elongated tube 10, which may also include a protective stubbed gasket-shape or boxed-shaped apparatus. The elongated tube 10 includes an inner surface, an outer surface, a first rim forming an entrance and a second rim forming an exit. In case of the boxed, a closed second rim. A length of the elongated tube 10 is from the first rim to the second rim. Tape 38, such as hypoallergenic tape, attaches the elongated tube 10 to the user's skin at an entry point of the fluid conducting lines 30. A first side edge and a second side edge are formed along the length of the elongated tube 10, forming a slit 26. The first side edge and the second side edge are biased to overlap one another. A disinfectant layer 28 such as an anti-bacterial, anti-viral, and/or anti-septic, is disposed on the inner surface of the elongated tube 10.

The elongated tube 10 includes a disinfectant layer 28 disposed on the inner surface to kill bacteria or viruses that could potentially contaminate the entry point of the fluid conducting lines 30. The disinfectant layer 28 may include, but is not limited to, isopropyl alcohol, chlorhexidine, povidone, hydrogen peroxide or any combination thereof. In certain embodiments, the elongated tube 10 may include a disinfectant cap 48 secured to the second rim. Pressure may be applied to the disinfectant cap 48, which disperses disinfectant into the elongated tube 10. The disinfectant cap 48 is removed and the elongated tube 10 is placed over the fluid conducting lines 30 via the slit 26.

In certain embodiments, the elongated tube 10 may include an accordion tube 12. The accordion tube 12 extends, retracts and bends into various positions. The accordion tube 12 may retain itself in the extended position and the retracted position. In certain embodiments, the accordion tube 12 may include straight portions 12a and accordion portions 12b. The straight portions 12a may include the disinfectant layer 28.

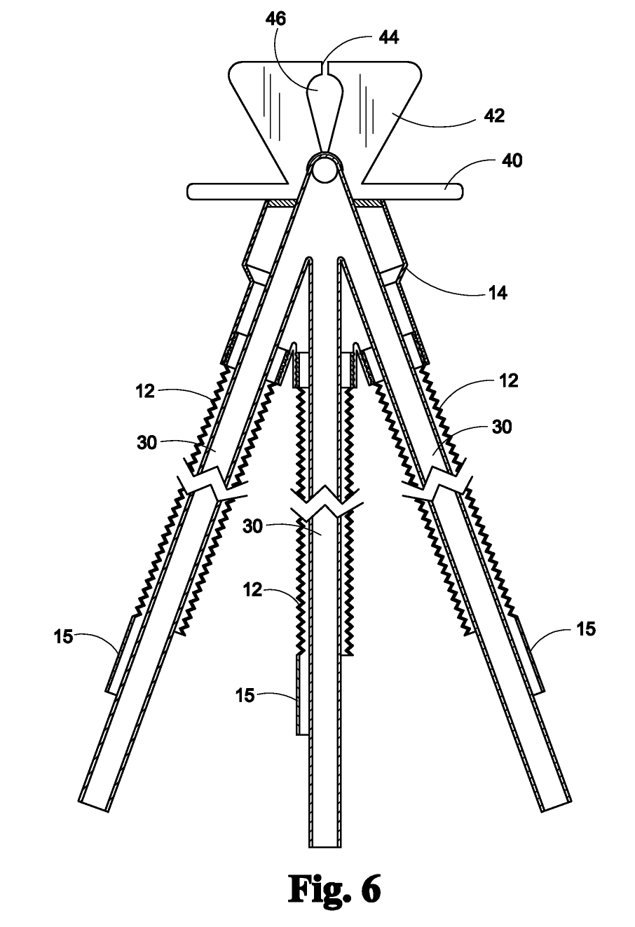

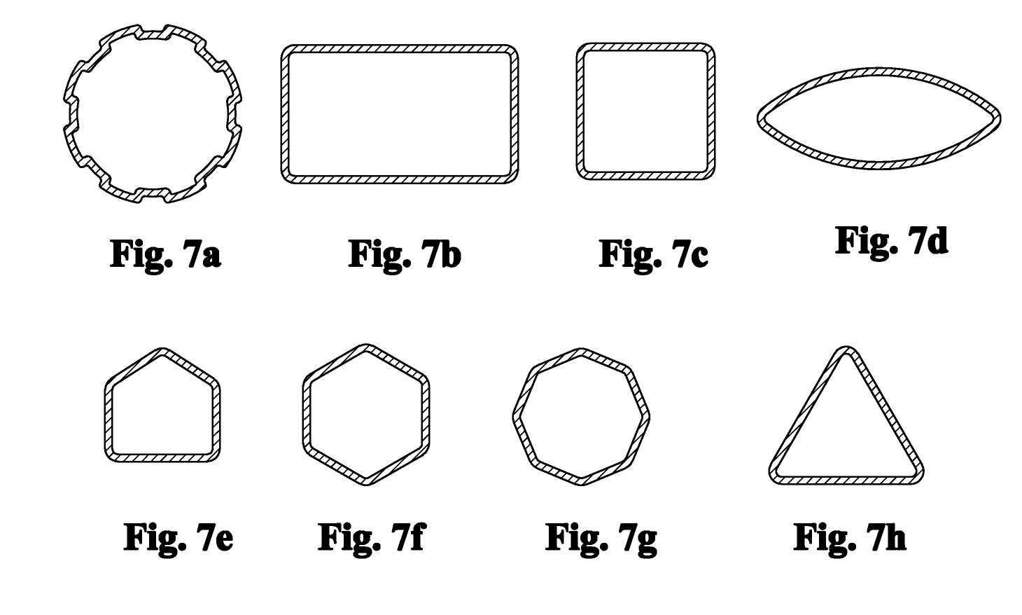

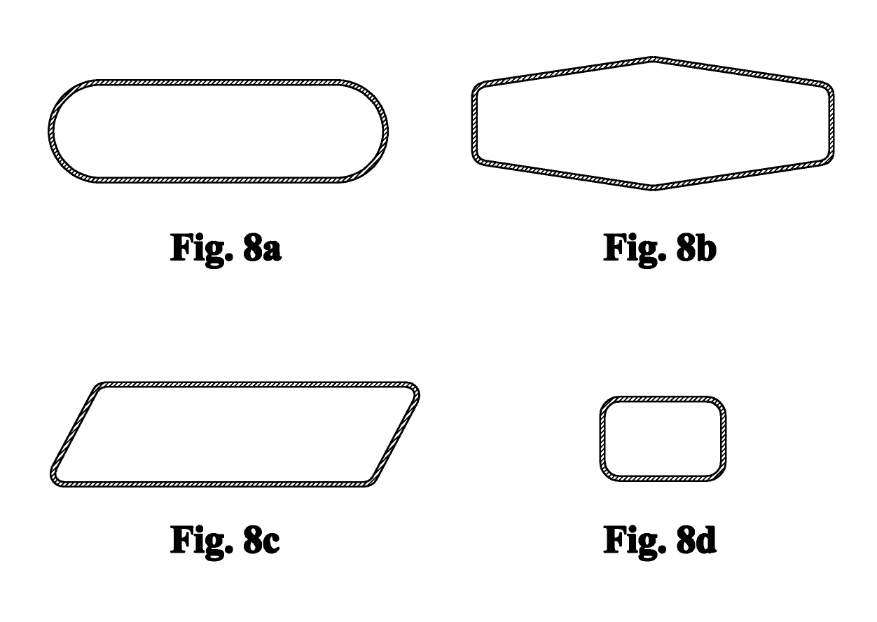

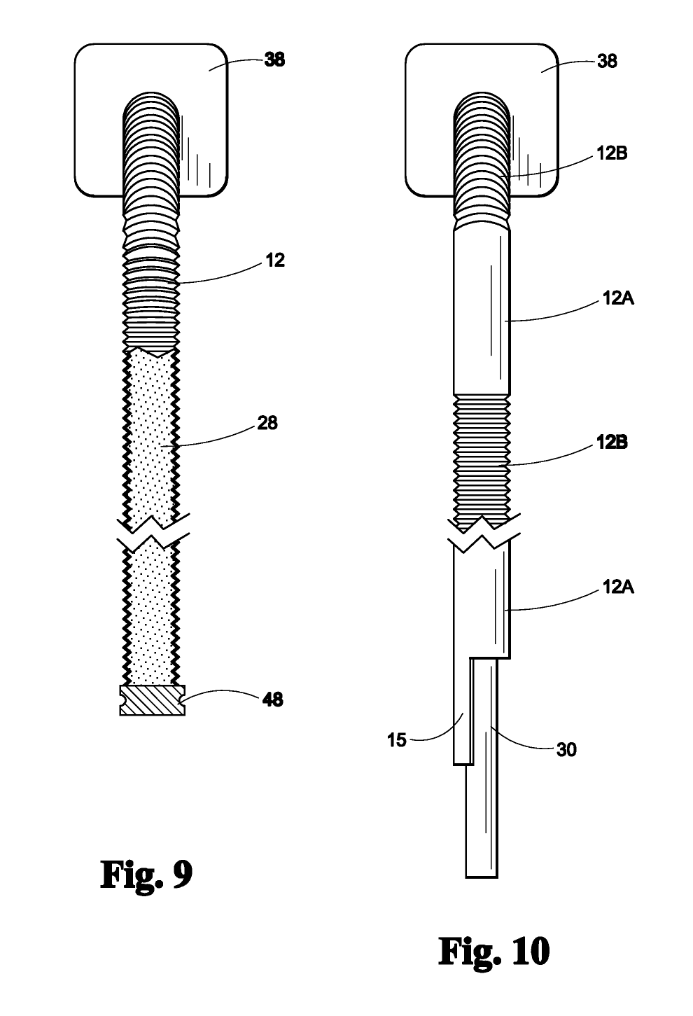

The elongated tube 10 may further include an entry tube 14 coupled to the accordion tube 12 by a circular coupler such as 20 or polygonal couplers in case of luminal structures shown in FIG. 7a through to FIG. 8d. The entry tube 14 may include an upper portion 16 and a lower portion 18. The upper portion 16 may include a larger diameter than the lower portion 18. The upper portion 16 may taper into the lower portion 18.

In certain embodiments, the entry tube 14 includes a first mating coupler 22a. The first mating coupler 22a may extend from the inner surface of the elongated tube 10. An adapter head 24 includes a second mating coupler 22b. The second mating coupler 22b mates with the first mating coupler 22a, locking the adapter head 24 to the entry tube 14. For example, the second mating coupler 22b may be a pair of notches formed on an outer surface and the first mating coupler 22a may be a pair of protrusions in the inner surface that fit within the pair of notches. Alternatively, the mating couplers may be threaded female and male portions. The adapter head 24 may include a cone shaped entrance and may be used for alternate types of fluid conducting lines 30, such as foley catheters to prevent soiling, sloth and infectious biofilms at the proximal entry point.

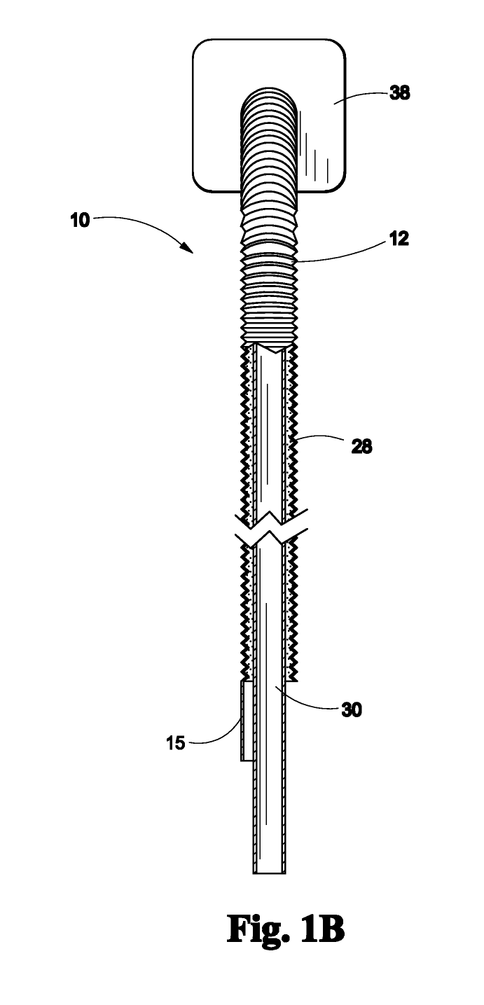

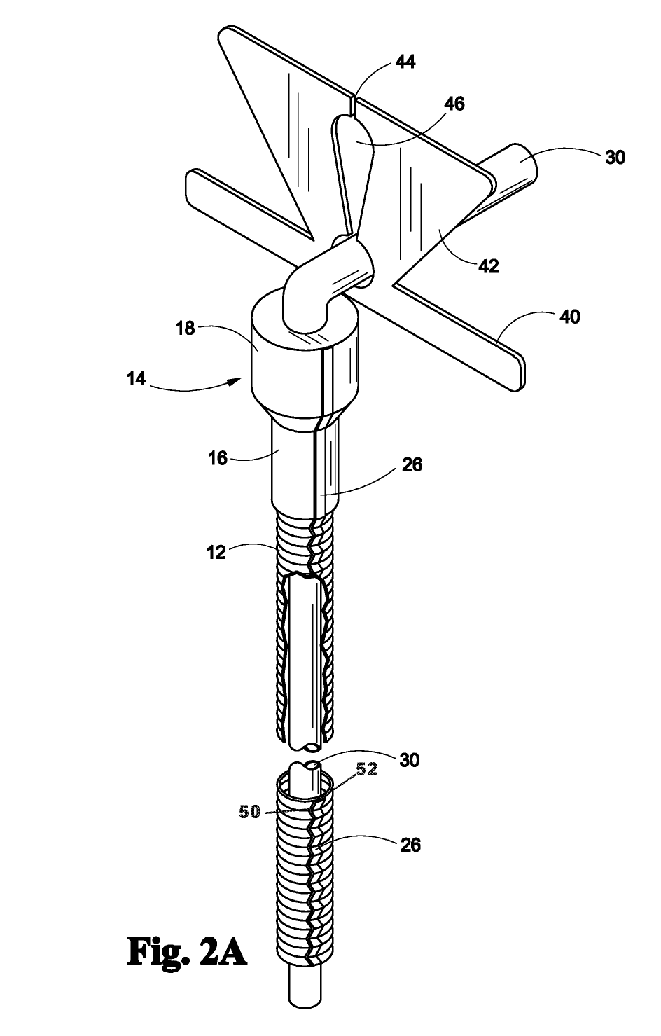

As illustrated in FIGS. 1A and 1B, the tape 38 may be sealed or inseparable from the first rim of the elongated tube 10. In such embodiments, the tape 38 includes an opening that aligns with the entrance of the elongated tube 10. The tape 38 may be sealed to the entire first rim and may be square shaped. Alternatively, as illustrated in FIGS. 2 and 3, the tape 38 may be sealed to a portion of the first rim by a sealant 36. In certain embodiments, the tape 38 may include a pair of arms 40 extending laterally from opposing sides and a body 42. The body 42 includes a slit 44 running from an edge of the body 42 into an opening 46 sized to receive and retain the fluid conducting line 30. The slit 44 may include a bulbous section to accommodate for different entry styles of various fluid conducting lines 30. The arms 40 wrap around the end of the elongated tube 10 and the body 42 adheres to a user's skin.

To further secure the elongated tube 10 to the fluid conducting lines 30, a distal tail appendage 15 may extend from the second rim of the elongated tube 10. The tail 15 may include healthcare state-of-the-art body-contacting materials such as a slender plastic, rubber, or processed gauze material substantially narrower than the elongated tube 10 and integral with the elongated tube 10. A second piece of tape may wrap around the tail 15 and the fluid conducting line 30, thereby further securing the elongated tube 10 to the fluid conducting line 30.

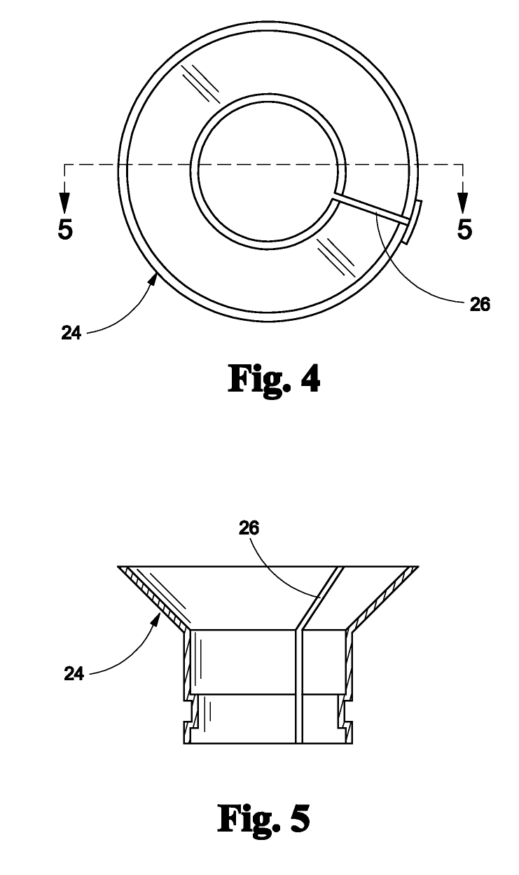

As illustrated in FIGS. 7a through 7g, the elongated tube 10 may be different shapes and sizes. The elongated tube 10 shape and sizes are chosen for the need of the particular fluid conducting line 30 to allow free fluid flow without any turbulence or constriction. In the order from the top upper left: circular periphery with indentations, a rectangular shape, a square, an ovoid a pentagon then a hexagonal shape and lastly another polygon. FIGS. 8a through 8d illustrate the different shapes and sizes of the entry tube 14. For example the entry tube 14 may include an extended width to accommodate for different sized fluid conducting lines 30. As illustrated in FIG. 6, the entry tube 14 may be sized to secure to a plurality of accordion tubes 12, such as three accordion tubes 12.

It should be understood, of course, that the foregoing relates to exemplary embodiments of the invention and that modifications may be made without departing from the spirit and scope of the invention as set forth in the following claims.

1. A protective barrier comprising:
an elongated tube comprising an inner surface, an outer surface, a first rim forming an entrance, a second rim forming an exit, and a length from the first rim to the second rim, wherein the elongated tube comprises an accordion tube operable to retain itself in an extended position and a retracted position;
a tape operable to secure the elongated tube to a skin of a user; and
a disinfectant layer disposed on the inner surface of the elongated tube.

2. (canceled)

3. The protective barrier of claim 1, wherein the elongated tube further comprises an entry tube coupled to the accordion tube.

4. The protective barrier of claim 3, wherein the entry tube comprises an upper portion and a lower portion, wherein the upper portion comprises a larger diameter than the lower portion.

5. The protective barrier of claim 4, wherein the upper portion tapers into the lower portion.

6. The protective barrier of claim 4, wherein the upper portion comprises a first mating coupler.

7. The protective barrier of claim 6, further comprising an adapter head secured to the first rim of the elongated tube by a second mating coupler of the adapter head releasably attached to the first mating coupler, wherein the adapter head comprises a cone shaped entrance.

8. The protective barrier of claim 1, wherein the tape is sealed to at least a portion of the first rim.

9. The protective barrier of claim 8, wherein the tape is sealed along the entire first rim.

10. The protective barrier of claim 1, wherein the tape comprises a pair of arms extending laterally from opposing sides and a body, wherein the body comprises a slit running from an edge of the body to a fluid conducting line opening.

11. The protective barrier of claim 1, further comprising a tail extending from the second rim.

12. The protective barrier of claim 1, wherein the elongated tube comprises an entry tube and a plurality of accordion tubes extending from to the entry tube.

13. The protective barrier of claim 1, wherein the elongated tube comprises a first side edge and a second side edge formed along the length forming a slit.

14. The protective barrier of claim 13, wherein the first side edge and the second side edge are biased to overlap.
